(19) United States
(12) Patent Application Publication
(10) Pub. No.: US 2020/0268716 A1
(43) Pub. Date: Aug. 27, 2020

(54) LULICONAZOLE AS ANTI-ACANTHAMOEBA AGENT AND METHOD FOR PRODUCING THE SAME

(71) Applicants: Pola Pharma Inc., Tokyo (JP); Nihon Nohyaku Co., Ltd., Tokyo (JP)

(72) Inventors: Yoshiyuki MIYATA, Tokyo (JP); Takaaki MASUDA, Yokohama-shi, Kanagawa (JP)

(21) Appl. No.: 15/781,030

(22) PCT Filed: Jun. 28, 2016
(86) PCT No.: PCT/JP2016/003106

§ 371 Date: Jun. 1, 2018

(30) Foreign Application Priority Data
Dec. 4, 2015 (JP) 2015-237951

(51) Int. Cl. A61K 31/4178

(52) U.S. Cl. CPC A61K 31/4178; C07B 2200/13

(57) ABSTRACT

An anti-Acanthamoeba agent contains luliconazole crystal as an active ingredient. The luliconazole has a crystal habit in which a ratio of I (20-2) to a total sum of I (001), I (100), I (10-1), I (011), I (110), I (11-1), I (10-2), I (11-2), I (020), I (021), I (20-2), I (121), I (013), I (11-3), and I (221) is 12% or more and/or a ratio of I (10-2) to a total sum of I (001), I (100), I (10-1), I (011), I (110), I (11-1), I (10-2), I (11-2), I (020), I (021), I (20-2), I (121), I (013), I (11-3), and I (221) is 20% or more.

### TECHNICAL FIELD

The present invention relates to anti-Acanthamoeba agents useful against infections caused by Acanthamoebae as pathogens, such as keratopathy and encephalitis.

### BACKGROUND ART

Acanthamoebae, which are amoeboid protozoans as well as basically ingest bacteria and freely live, occasionally cause infections such as keratitis and encephalitis. At present, there is no drug effective against such infections caused by Acanthamoebae. When being serious, such infections may cause blindness and may result in death due to opportunistic infections in rare cases. Lactoferrin, lactoferricin, and the like, which have been reported as anti-Acanthamoeba agents in patent literatures, have had difficulty in exerting effects in practical application (see, for example, Patent Literature 1). Acanthamoebae includes several known subspecies such as A. castellanii and A. polypharga, all of which have the possibility of causing infections. In particular, acanthamebiasis caused by a contact lens has been greatly problematic in recent years. Furthermore, interactions between Acanthamoeba and HIV infection (see, for example, Non Patent Literature 1), and between Acanthamoeba and MRSA infection (see, for example, Non Patent Literature 2 and Non Patent Literature 3) have also been suspected, and the need to take countermeasures against the interactions has become urgent.

Luliconazole, which has been already marketed as an antifungal agent, has been already known, e.g., to have action against Trichomonas, and to have several crystal habits, resulting in different extents of action against Trichomonas (see, for example, Patent Literatures 2 to 9). There is also a literature insisting that there is another crystal system with regard to luliconazole crystals (see, for example, Patent Literature 10).

However, luliconazole has not been known to have anti-Acanthamoeba activity at all, and the anti-Acanthamoeba activity has not been known to differ according to a crystal habit at all either. In addition, a luliconazole crystal having a crystal habit in which (20-2) plane or (10-2) plane are specific growth planes has not been known at all either.

### CITATION LIST

### Patent Literature

Patent Literature 1: Japanese Patent Laid-Open No. 2011-246458
Patent Literature 2: Japanese Patent Laid-Open No. 2015-91890
Patent Literature 3: Japanese Patent Laid-Open No. 2015-63566
Patent Literature 4: Japanese Patent Laid-Open No. 2015-51945
Patent Literature 5: Japanese Patent Laid-Open No. 2015-27984
Patent Literature 6: Japanese Patent Laid-Open No. 2015-27983
Patent Literature 7: Japanese Patent Laid-Open No. 2015-27972
Patent Literature 8: Japanese Patent Laid-Open No. 2014-172858
Patent Literature 9: Japanese Patent Laid-Open No. 2014-74008
Patent Literature 10: Chinese Patent No. 103012385

### Non Patent Literature

Non Patent Literature 1: J. Clin. Microbiol. 2012, 50(3), 1128-31
Non Patent Literature 2: BMJ Case Rep. 2009, pii: bcr08. 2008. 0642
Non Patent Literature 3: Environ. Microbiol. 2006, 8(6), 1130-3

### SUMMARY OF INVENTION

### Technical Problem

The present invention was made under such circumstances with an object to provide a novel anti-Acanthamoeba agent.

### Solution to Problem

The present inventors persevered in intensive research efforts for a novel anti-Acanthamoeba agent with respect to such circumstances, and found that luliconazole has anti-Acanthamoeba activity, and the invention was thus accomplished. By further examination, it was found that a crystal having a crystal habit in which (20-2) plane and/or (10-2) plane are specific growth planes is particularly excellent in such action, and the invention was further expanded. In other words, the present invention is as described below.

<1> An anti-Acanthamoeba agent, comprising luliconazole as an active ingredient.

<2> The anti-Acanthamoeba agent according to <1>, wherein the luliconazole is present as a solid in the agent.

<3> The anti-Acanthamoeba agent according to <1> or <2>,

wherein the luliconazole has a crystal habit in which

a ratio of I (20-2) to a total sum of I (001), I (100), I (10-1), I (011), I (110), I (11-1), I (10-2), I (11-2), I (020), I (021), I (20-2), I (121), I (013), I (11-3), and I (221) is 12% or more,

provided that in diffraction peaks detected in a range of 2θ=5 to 35° in a powder X-ray diffraction pattern with CuKα as a radiation source, peak intensities at diffraction peaks corresponding to (001) plane, (100) plane, (10-1) plane, (011) plane, (110) plane, (11-1) plane, (10-2) plane, (11-2) plane, (020) plane, (021) plane, (20-2) plane, (121) plane, (013) plane, (11-3) plane, and (221) plane are I (001), I (100), I (10-1), I (011), I (110), I (11-1), I (10-2), I (11-2), I (020), I (021), I (20-2), I (121), I (013), I (11-3), and I (221), respectively.

<4> The anti-Acanthamoeba agent according to any one of <1> to <3>,

wherein the luliconazole has a crystal habit in which
a ratio of I (10-2) to a total sum of I (001), I (100), I (10-1), I (011), I (110), I (11-1), I (10-2), I (11-2), I (020), I (021), I (20-2), I (121), I (013), I (11-3), and I (221) is 20% or more,
provided that in diffraction peaks detected in a range of 2θ=5 to 35° in a powder X-ray diffraction pattern with CuKα as a radiation source, peak intensities at diffraction peaks corresponding to (001) plane, (100) plane, (10-1) plane, (011) plane, (110) plane, (11-1) plane, (10-2) plane, (11-2) plane, (020) plane, (021) plane, (20-2) plane, (121) plane, (013) plane, (11-3) plane, and (221) plane are I (001), I (100), I (10-1), I (011), I (110), I (11-1), I (10-2), I (11-2), I (020), I (021), I (20-2), I (121), I (013), I (11-3), and I (221), respectively.

<5> A luliconazole crystal, having a crystal habit in which

a ratio of I (20-2) to a total sum of I (001), I (100), I (10-1), I (011), I (110), I (11-1), I (10-2), I (11-2), I (020), I (021), I (20-2), I (121), I (013), I (11-3), and I (221) is 12% or more,

provided that in diffraction peaks detected in a range of 2θ=5 to 35° in a powder X-ray diffraction pattern with CuKα as a radiation source, peak intensities at diffraction peaks corresponding to (001) plane, (100) plane, (10-1) plane, (011) plane, (110) plane, (11-1) plane, (10-2) plane, (11-2) plane, (020) plane, (021) plane, (20-2) plane, (121) plane, (013) plane, (11-3) plane, and (221) plane are I (001), I (100), I (10-1), I (011), I (110), I (11-1), I (10-2), I (11-2), I (020), I (021), I (20-2), I (121), I (013), I (11-3), and I (221), respectively.

<6> A luliconazole crystal, having a crystal habit in which

a ratio of I (10-2) to a total sum of I (001), I (100), I (10-1), I (011), I (110), I (11-1), I (10-2), I (11-2), I (020), I (021), I (20-2), I (121), I (013), I (11-3), and I (221) is 20% or more,

provided that in diffraction peaks detected in a range of 2θ=5 to 35° in a powder X-ray diffraction pattern with CuKα as a radiation source, peak intensities at diffraction peaks corresponding to (001) plane, (100) plane, (10-1) plane, (011) plane, (110) plane, (11-1) plane, (10-2) plane, (11-2) plane, (020) plane, (021) plane, (20-2) plane, (121) plane, (013) plane, (11-3) plane, and (221) plane are I (001), I (100), I (10-1), I (011), I (110), I (11-1), I (10-2), I (11-2), I (020), I (021), I (20-2), I (121), I (013), I (11-3), and I (221), respectively.

<7> A luliconazole crystal, having a crystal habit in which

a ratio of I (20-2) and a ratio of I (10-2) to a total sum of I (001), I (100), I (10-1), I (011), I (110), I (11-1), I (10-2), I (11-2), I (020), I (021), I (20-2), I (121), I (013), I (11-3), and I (221) is 12% or more and 20% or more, respectively,

provided that in diffraction peaks detected in a range of 2θ=5 to 35° in a powder X-ray diffraction pattern with CuKα as a radiation source, peak intensities at diffraction peaks corresponding to (001) plane, (100) plane, (10-1) plane, (011) plane, (110) plane, (11-1) plane, (10-2) plane, (11-2) plane, (020) plane, (021) plane, (20-2) plane, (121) plane, (013) plane, (11-3) plane, and (221) plane are I (001), I (100), I (10-1), I (011), I (110), I (11-1), I (10-2), I (11-2), I (020), I (021), I (20-2), I (121), I (013), I (11-3), and I (221), respectively.

<8> A method for producing the luliconazole crystal as defined in any one of <5> to <7>, the method comprising recrystallizing luliconazole with acetonitrile that may contain a polar solvent.

<9> The production method according to <8>, wherein the polar solvent is selected from water, methanol, ethanol, ethyl acetate, acetone, dimethylsulfoxide, and dimethylformamide.

<10> A method for producing the luliconazole crystal as defined in any one of <5> to <7>, the method comprising recrystallizing luliconazole with a mixed solvent of an aprotic polar solvent and a protic polar solvent.

<11> The production method according to any one of <8> to <10>, wherein a solvent selected from normal hexane, cyclohexane, and petroleum ether is used as a poor solvent.

### Advantageous Effects of Invention

In accordance with the present invention, a novel anti-Acanthamoeba agent can be provided.

### BRIEF DESCRIPTION OF DRAWINGS

FIG. 1 is a view showing the measured X-ray powder diffraction data of crystal in Example 2.

FIG. 2 is a view showing the calculated values of the powder pattern of the crystal of the present invention in a range of 2θ=5 to 35°, obtained using the single-crystal X-ray diffraction data of luliconazole.

FIG. 3 illustrates the microphotographs of crystals in Examples 2 to 6 (photographs substituted for the drawing). FIG. 3(a) illustrates the crystal in Example 2, FIG. 3(b) illustrates the crystal in Example 3, FIG. 3(c) illustrates the crystal in Example 4, FIG. 3(d) illustrates the crystal in Example 5, and FIG. 3(e) illustrates the crystal in Example 6.

### DESCRIPTION OF EMBODIMENTS

(1) Active Ingredient

An anti-Acanthamoeba agent of the present invention comprises luliconazole as an active ingredient. Luliconazole, having a structure described below, has been already marketed and used as an antifungal agent.

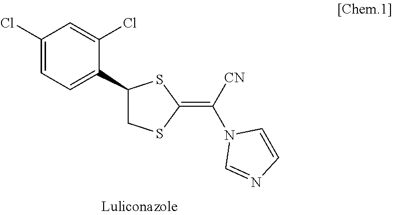 [SMILES: N#C/C(=C1/SC[C@@H](c2ccc(Cl)cc2Cl)S1)n1ccnc1]

Such luliconazole can be synthesized according to a method described, for example, in Japanese Patent Laid-Open No. 60-218387. In other words, such a compound represented by the general formula (1) can be obtained by allowing 1-cyanomethylimidazole to react with carbon disulfide to obtain a compound (III), which is allowed to react with a compound of the general formula (II) having leaving groups. After the reaction, the compound may be purified by a recrystallization method as a usual method.

In the formulae described below, R and X represent a hydrogen atom or a halogen atom. In the compound represented by the general formula (1), a compound corresponding to R═X═Cl is luliconazole. Preferred examples of such leaving groups Y and Y′ include methanesulfonyloxy group, benzenesulfonyloxy group, p-toluenesulfonyloxy group, or a halogen atom (see the following scheme).

Luliconazole produced in such steps is recrystallized and purified with an alcohol that may contain a polar solvent, to obtain a monoclinic crystal that has space group P2₁, with lattice parameters of a=9.0171(9) Å, b=8.167(1) Å, c=10.878(1) Å, and β=95.917(9) °.

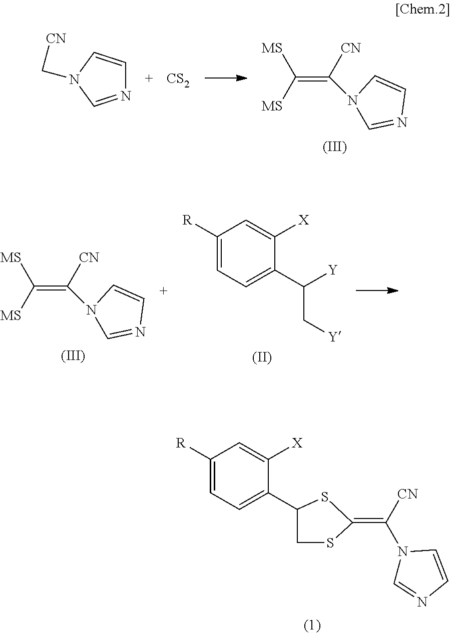

In the formulae, Y and Y′ represent leaving groups, and M represents an alkali metal atom.

(2) Luliconazole Crystal

Luliconazole exhibits excellent anti-Acanthamoeba activity. The anti-Acanthamoeba agent of the present invention can be applied, without particular limitation, to microorganisms that are classified into Acanthamoebae and are responsible for diseases such as keratitis. The anti-Acanthamoeba agent of the present invention well inhibits the growth of the microorganisms. Against Acanthamoebae, luliconazole exhibits anti-Acanthamoeba activity even when being solid, i.e., a crystal in itself, and exhibits a similar effect even when being in a solution state. Luliconazole is difficult to dissolve in an aqueous carrier, and a solvent such as ethanol, benzyl alcohol, N-alkylpyrrolidone, or diisopropyl adipate is required for locally administering homogeneously dissolved luliconazole to the affected area. In consideration of application to keratitis, the use of such a solvent is unfavorable because of resulting in the danger of damage to the cornea. Thus, in the case of local administration of the anti-Acanthamoeba agent of the present invention, the administration of the anti-Acanthamoeba agent that is solid is preferable. A dispersion liquid preparation in which a bulk powder is homogeneously dispersed in an aqueous medium that scarcely causes corneal disorders, or an ophthalmic ointment preparation in which such a bulk powder is homogeneously dispersed in an ointment base such as vaseline is preferred as a preparation for administration of such a solid against such a disease, i.e., a preparation in which luliconazole in solid (i.e., crystal) form is present in an agent (hereinafter also referred to as solid preparation). In such a solid preparation for local administration in solid form, it is preferable to use a crystal in which (20-2) plane and/or (10-2) plane are specific growth planes.

The specific growth plane of a crystal is a plane belonging to a peak of which the peak intensity is significantly great compared with the total sum of the peak intensities of the other diffraction peaks in the range of measured diffraction angles in the case of performing powder X-ray diffractometry. In other words, the specific growth plane of a crystal can be detected, for example, as a peak of which the peak intensity of the diffraction peak is specifically high in the powder X-ray diffractometry of the crystal.

A crystal in which (20-2) plane is a specific growth plane has a specific peak in the vicinity of 2θ=24.4° in a powder X-ray diffraction pattern with CuKα as a radiation source.

A value of 2θ in the vicinity of 24.4° is, for example, in a range of 24.4°±0.7° preferably 24.4°±0.5°.

A crystal in which (10-2) plane is a specific growth plane has a specific peak in the vicinity of 2θ=18.3° in a powder X-ray diffraction pattern with CuKα as a radiation source.

A value of 2θ in the vicinity of 18.3° is, for example, in a range of 18.3°±0.7° preferably 18.3°±0.5°.

In a case in which the peak intensity of a diffraction peak corresponding to (20-2) plane is specifically high, the value of I (20-2)/[I (001)+I (100)+I (10-1)+I (011)+I (110)+I (11-1)+I (10-2)+I (11-2)+I (020)+I (021)+I (20-2)+I (121)+I (013)+I (11-3)+I (221)]×100 is preferably 12% or more, more preferably 15% or more, and still more preferably 19% or more, with the proviso that in diffraction peaks detected in a range of 2θ=5 to 35° in a powder X-ray diffraction pattern with CuKα as a radiation source, the peak intensities of diffraction peaks corresponding to (001) plane, (100) plane, (10-1) plane, (011) plane, (110) plane, (11-1) plane, (10-2) plane, (11-2) plane, (020) plane, (021) plane, (20-2) plane, (121) plane, (013) plane, (11-3) plane, and (221) plane are I (001), I (100), I (10-1), I (011), I (110), I (11-1), I (10-2), I (11-2), I (020), I (021), I (20-2), I (121), I (013), I (11-3), and I (221), respectively. The upper limit of the value is presumed to be around 50% in view of practical maintenance of the properties of a crystal.

In a case in which the peak intensity of a diffraction peak corresponding to (10-2) plane is specifically high, the value of I (10-2)/[I (001)+I (100)+I (10-1)+I (011)+I (110)+I (11-1)+I (10-2)+I (11-2)+I (020)+I (021)+I (20-2)+I (121)+I (013)+I (11-3)+I (221)]×100 is preferably 20% or more, more preferably 25% or more, and still more preferably 30% or more. The upper limit of the value is presumed to be around 50% in view of practical maintenance of the properties of a crystal.

The action of inhibiting the growth of Acanthamoeba is increased with increasing the peak intensity ratios. A particularly preferred embodiment is a crystal having both crystal habit properties in which (20-2) plane and (10-2) plane specifically grow.

In order to obtain crystals having such a crystal habit, it is preferable to recrystallize monoclinic crystals of luliconazole obtained by the above-described method or the like, using acetonitrile that may contain a polar solvent. The recrystallization, which can be performed according to a usual method except for selection of the solvent, is preferably performed, for example, by dissolving the luliconazole crystals in the solvent under warming (ordinary at 40 to 80° C., preferably 40 to 60° C.) to completely dissolve the crystal in the solvent, followed by cooling the resultant under stirring (ordinary at 5° C. to room temperature), filtering the crystals, and drying the crystals by sending air at a low temperature of room temperature to around 40° C. The recrystallization can also be performed by completely dissolving the crystals in the solvent, followed by evaporating the solvent. Preferred examples of the polar solvent include water, methanol, ethanol, ethyl acetate, acetone, dimethylsulfoxide, and dimethylformamide, and a polar solvent selected from water, methanol, ethanol, ethyl acetate, dimethylsulfoxide, and dimethylformamide is more preferred.

When acetonitrile contains a polar solvent in the production method, the amount of the added polar solvent is ordinarily 5 to 95 mass %, preferably 7 to 75 mass %, and more preferably 7 to 20 mass % with respect to the mass of the acetonitrile.

A method for recrystallizing luliconazole crystals with the mixed solvent of an aprotic polar solvent and a protic polar solvent can also be used in the method for producing a crystal in which (20-2) plane and/or (10-2) plane are specific growth planes. Preferred examples of the aprotic polar solvent include ethyl acetate, acetone, dimethylsulfoxide, and dimethylformamide. Preferred examples of the protic polar solvent include water, methanol, and ethanol. The mixture ratio (mass ratio) between the aprotic polar solvent and the protic polar solvent can be selected in consideration of the solubility of the crystals, the boiling point of the solvent, and the like, and is ordinarily 9:1 to 1:9, preferably 7:3 to 3:7, more preferably 3:2 to 2:3, and still more preferably 4:3 to 3:4. The step of the recrystallization can be performed in a manner similar to the recrystallization using the acetonitrile.

In order to promote crystal precipitation, a nonpolar solvent such as normal hexane, cyclohexane, or petroleum ether can also be added as a poor solvent. In such a case, the amount of the added poor solvent is ordinarily appropriately 5 to 60 mass %, preferably 7 to 50 mass %, and more preferably 10 to 40 mass % with respect to the total sum of the other solvents.

The preferred average particle diameter of the crystals used in the present invention is 10 to 500 μm, more preferably 50 to 400 μm, and still more preferably 150 to 300 μm. This is because the too small average particle diameter may unfavorably result in a small crystal habit while the too large average particle diameter may result in constitution of an obstacle to administration. The crystals satisfying such an average particle diameter are obtained, for example, by such a method as described above. The average particle diameter of luliconazole can be measured and obtained as a number average particle diameter. The number average particle diameter can be measured and obtained as the average diameter of particles by analyzing a microscope image. For example, the measurement is performed in the following procedure. First, a powder of luliconazole is observed using, as an inverted microscope, an inverted microscope Diaphot manufactured by NIKON CORPORATION. Then, optional particles are selected, and the particle diameters of the particles are measured. In such a case, the measurement is performed for 100 or more particles. Further, the average particle diameter of luliconazole can be obtained as an average particle diameter measured and obtained with a laser diffraction-type particle size distribution measuring apparatus.

Luliconazole crystals obtained by, for example, recrystallization and/or the like may be ground in a wet or dry process by a usual method in order to adjust the average particle diameter of the crystals, and preferred examples of instruments used in the grinding include jet mills, dinomills, cobol mills, mortar machines, and ball mills. In the case of performing dry grinding with a jet mill, it is preferable to perform coarse grinding with a speed mill and/or the like in advance, and to perform treatment. In this case, it is also possible to add an excipient such as starch or cellulose, a lubricant such as magnesium stearate, and/or the like, and to perform the treatment. In the case of performing wet grinding with a medium mill such as a dinomill or a cobol mill, it is preferable to perform grinding with a zirconia or titanium medium using an aqueous medium that may contain an alcohol and/or the like, to then remove the solid grinding medium by filtration and/or the like, and to then remove the liquid medium by, e.g., distillation off under reduced pressure. In some cases, it is also possible to add a surfactant to improve the wettability of luliconazole for the liquid medium. Further, it is preferable to operate the mortar machine provided with an agate mortar and a pestle. As a matter of course, it is also possible to perform grinding by manual operation with a mortar and a pestle. A ball mill may be used in wet or dry grinding.

(3) Anti-Acanthamoeba Agent

The anti-Acanthamoeba agent of the present invention may have an embodiment in which the agent consists of luliconazole, or may have an embodiment in which the agent further contains, e.g., an optional ingredient that is ordinarily used for preparation unless interfering with the effects of the present invention. One preferred embodiment of the anti-Acanthamoeba agent of the present invention is a preparation in which solid luliconazole is present in the agent (solid preparation). Examples of the solid preparation include: solid preparations such as tablets, capsules, and powders; semisolid preparations such as ointments and creams; and solid dispersion liquid preparations in which a solid is dispersed in an aqueous medium. There can be preferably described an example in which the anti-Acanthamoeba agent of the present invention is prepared into: an orally-administered agent such as a tablet or a capsule; an external preparation such as an ointment, a cream, or a solid dispersion liquid preparation, together with an optional ingredient such as an excipient, a disintegrant, a binder, a flavoring agent, a dispersant, an emulsifier, or an aqueous medium; a powder, together with an optional ingredient such as an extender; or the like. Particularly preferred examples include a directly intraocularly administered powder, an aqueous medium dispersion preparation, or an ophthalmic ointment in ointment form.

In a case in which the anti-Acanthamoeba agent of the present invention is processed into an anti-Acanthamoeba composition such as a medicine, the content of luliconazole which is an active ingredient is ordinarily 0.1 to 50 mass % and preferably 0.5 to 15 mass % with respect to the total amount of the composition.
The anti-Acanthamoeba agent of the present invention can be used for preventive and therapeutic applications against infections caused by Acanthamoeba, for example, keratitis. The dosage form of the anti-Acanthamoeba agent of the present invention can be selected as appropriate in consideration of the body weight, age, sex, symptom, and/or the like of a patient, and in the case of an adult, it is preferable to administer the preparation with the active ingredient in an amount of ordinarily 1 to 100 mg daily. It is preferable to perform the administration once daily or twice daily.

### EXAMPLES

The present invention is explained in more detail below by describing examples, but the present invention is not limited to the examples.

### Example 1

Acanthamoeba polyphaga (A. polyphaga; clinical isolate) was cultured in an ATCC712 medium (hereinafter simply referred to as medium) at 25° C. for 1 week, and the medium was adjusted by adding a medium so that the number of the amoebae was 100,000 amoebae/mL, to make a preculture liquid. A control and samples were prepared by adding 4.3 mL of medium, 200 μL of specimen, and 500 μL of preculture liquid. The amoebae were cultured at 25° C. for 72 hours, and the number of the amoebae was counted by a Neubauer counting chamber.

Luliconazole used in the specimen was obtained as described below. A compound was synthesized according to a method described in Japanese Patent Laid-Open No. 60-218387, an ethyl acetate/normal hexane mixture (5:1) was added, and the resultant was warmed at 60° C. and dissolved under stirring. Crystals were precipitated while stirring the resultant in cooling water at 5° C., were filtered, and dried by sending air at 40° C. for 48 hours to obtain the crystals.

The specimen was prepared using methanol as a solvent so that the final concentration of luliconazole was 0, 5, 10, 20, or 40 μg/mL. The specimen in which the final concentration of luliconazole was 0 μg/mL was used as the control.

Growth inhibition rates were determined based on [(the number of amoebae in control−the number of amoebae in each sample)/the number of amoebae in control]×100. MIC and IC₅₀ were calculated based on the growth inhibition rates by a usual method. The growth inhibition rates were listed in Table 1. MIC was 40 μg/mL, and IC₅₀ was 20 μg/mL.

TABLE 1

| | | |
|---|---|---|
| Concentration | Inhibition Rate (%) | SD |

| | | | |
|---|---|---|---|
| 40 | μg/mL | 95.0 | 8.7 |
| 20 | μg/mL | 50.0 | 22.9 |
| 10 | μg/mL | 40.0 | 0 |
| 5 | μg/mL | 20.0 | 8.7 |

### Example 2

In order to produce a solid preparation, crystals having a crystal habit were produced on the following conditions.

(Solvent) Acetonitrile

(Crystallization Method) Evaporation method (Luliconazole was dissolved at 50° C., and cooled to room temperature. Then, when residual crystals were present, the crystals were removed. The solvent was evaporated at room temperature for 1 day to result in recrystallization, and filtration and air drying were performed.)

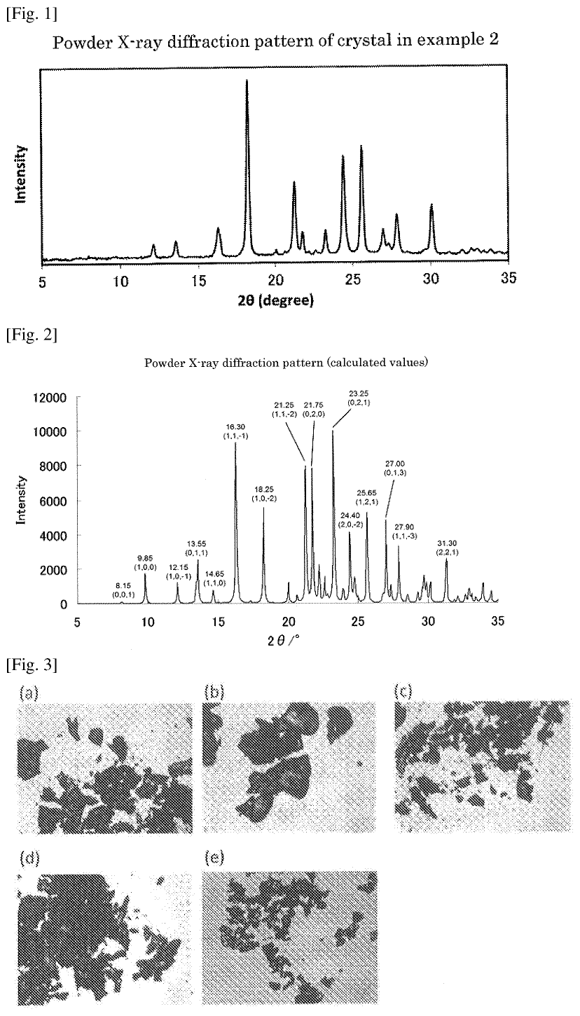

The powder X-ray diffractometry (apparatus model name: XRD-DSCII; manufacturer name: Rigaku Corporation; conditions: X-ray source: CuKα; measurement temperature: room temperature; tube voltage: 40 kV; tube current: 40 mA; 2θ: 5 to 35°; step angle: 0.05°) of the obtained crystals revealed that a diffraction pattern as listed in Table 2 below was shown. Further, the results of the powder X-ray diffractometry are shown in FIG. 1. It is revealed that the crystals were the luliconazole crystals of the present invention. A crystal plane corresponding to each diffraction peak was identified by comparison with data from single-crystal X-ray structure analysis (apparatus model name: RU-H2R; manufacturer name: Rigaku Corporation; conditions: X-ray source: CuKα; measurement temperature: 26° C.; tube voltage: 50 kV; tube current: 180 mA; 2θmax: 150.0°; structure analysis method: direct method (SHELX86)). The various data obtained from the single-crystal X-ray structure analysis are shown below and in FIG. 2. Specifically growing crystal planes were specified from the ratios of diffraction peak intensities by comparing the data with the powder X-ray diffractometry data. The results are listed in Table 2.

Each peak intensity ratio is a ratio with respect to the total sum of the peak intensities of diffraction peaks corresponding to (001) plane, (100) plane, (10-1) plane, (011) plane, (110) plane, (11-1) plane, (10-2) plane, (11-2) plane, (020) plane, (021) plane, (20-2) plane, (121) plane, (013) plane, (11-3) plane, and (221) plane.

Crystal system: monoclinic

Space group: P2₁

Lattice parameters

a=9.0171(9) Å

b=8.167(1) Å

c=10.878(1) Å

β=95.917(9°)

R factors

R=0.046

R_{w}=0.047

TABLE 2

| | Diffraction Peak | | | | |
|---|---|---|---|---|---|
| | Calculated Value* | | Crystal in Example 2 | | |
| | | Intensity | | Intensity | |
| | Intensity | Ratio | Intensity | Ratio | |
| Plane | (cps) | <A> (%) | (cps) | <B> (%) | B/A |
| (0, 0, 1) | 108 | 0.2 | 330 | 0.2 | 1.0 |
| (1, 0, 0) | 1741 | 2.6 | 393 | 0.2 | 0.1 |
| (1, 0, −1) | 1228 | 1.8 | 4192 | 2.0 | 1.1 |
| (0, 1, 1) | 2542 | 3.8 | 5295 | 2.5 | 0.7 |
| (1, 1, 0) | 765 | 1.1 | 305 | 0.1 | 0.1 |
| (1, 1, −1) | 9316 | 13.9 | 9642 | 4.6 | 0.3 |
| (1, 0, −2) | 5518 | 8.2 | 57877 | 27.4 | 3.3 |
| (1, 1, −2) | 7951 | 11.9 | 24532 | 11.6 | 1.0 |
| (0, 2, 0) | 7835 | 11.7 | 7930 | 3.7 | 0.3 |
| (0, 2, 1) | 10000 | 14.9 | 8663 | 4.1 | 0.3 |
| (2, 0, −2) | 4131 | 6.2 | 32785 | 15.5 | 2.5 |
| (1, 2, 1) | 5251 | 7.8 | 36130 | 17.1 | 2.2 |
| (0, 1, 3) | 4806 | 7.2 | 8633 | 4.1 | 0.6 |
| (1, 1, −3) | 3305 | 4.9 | 13700 | 6.5 | 1.3 |
| (2, 2, 1) | 2560 | 3.8 | 1077 | 0.5 | 0.1 |

*Calculated value of powder X-ray diffraction pattern calculated from single-crystal structure analysis data

### Example 3

Dissolution was performed with an ethyl acetate/normal hexane mixture (4:3) as a recrystallization solvent under reflux for 2 hours, the residue was removed by filtration, ice cooling under stirring was carried out to perform recrystallization, and filtration and air drying were performed for 4 hours to obtain crystals which are the anti-Acanthamoeba agent of the present invention. Powder X-ray analysis was performed in like manner with Example 2. The X-ray powder diffraction data of the resultant is as described below.

TABLE 3

| | Diffraction Peak | | | | |
|---|---|---|---|---|---|
| | Calculated Value* | | Crystal in Example 3 | | |
| | | Intensity | | Intensity | |
| | Intensity | Ratio | Intensity | Ratio | |
| Plane | (cps) | <A> (%) | (cps) | <B> (%) | B/A |
| (0, 0, 1) | 108 | 0.2 | 26 | 2.1 | 10.5 |
| (1, 0, 0) | 1741 | 2.6 | 22 | 1.8 | 0.7 |
| (1, 0, −1) | 1228 | 1.8 | 23 | 1.8 | 1.0 |
| (0, 1, 1) | 2542 | 3.8 | 22 | 1.8 | 0.5 |
| (1, 1, 0) | 765 | 1.1 | 19 | 1.5 | 1.4 |
| (1, 1, −1) | 9316 | 13.9 | 754 | 60.6 | 4.4 |
| (1, 0, −2) | 5518 | 8.2 | 21 | 1.7 | 0.2 |
| (1, 1, −2) | 7951 | 11.9 | 71 | 5.7 | 0.5 |
| (0, 2, 0) | 7835 | 11.7 | 25 | 2.0 | 0.2 |
| (0, 2, 1) | 10000 | 14.9 | 106 | 8.5 | 0.6 |
| (2, 0, −2) | 4131 | 6.2 | 25 | 2.0 | 0.3 |
| (1, 2, 1) | 5251 | 7.8 | 32 | 2.6 | 0.3 |
| (0, 1, 3) | 4806 | 7.2 | 53 | 4.3 | 0.6 |
| (1, 1, −3) | 3305 | 4.9 | 27 | 2.2 | 0.4 |
| (2, 2, 1) | 2560 | 3.8 | 18 | 1.4 | 0.4 |

*Calculated value of powder X-ray diffraction pattern calculated from single-crystal structure analysis data

### Example 4

Dissolution was performed with a methanol/dimethylsulfoxide mixture (1:1) as a recrystallization solvent at 50° C. Then, when residual crystals were present, the crystals were removed. Cooling at 5° C. was carried out to perform recrystallization, and filtration and air drying were performed to obtain crystals which were the anti-Acanthamoeba agent of the present invention. Powder X-ray analysis was performed in like manner with Example 2. The X-ray powder diffraction data of the resultant is as described below.

TABLE 4

| | Diffraction Peak | | | | |
|---|---|---|---|---|---|
| | Calculated Value* | | Crystal in Example 4 | | |
| | | Intensity | | Intensity | |
| | Intensity | Ratio | Intensity | Ratio | |
| Plane | (cps) | <A> (%) | (cps) | <B> (%) | B/A |
| (0, 0, 1) | 108 | 0.2 | −30 | 0.0 | 0.0 |
| (1, 0, 0) | 1741 | 2.6 | 292 | 0.2 | 0.1 |
| (1, 0, −1) | 1228 | 1.8 | 2103 | 1.7 | 0.9 |
| (0, 1, 1) | 2542 | 3.8 | 2322 | 1.9 | 0.5 |
| (1, 1, 0) | 765 | 1.1 | 132 | 0.1 | 0.1 |
| (1, 1, −1) | 9316 | 13.9 | 8860 | 7.2 | 0.5 |
| (1, 0, −2) | 5518 | 8.2 | 30002 | 24.3 | 3.0 |
| (1, 1, −2) | 7951 | 11.9 | 15747 | 12.8 | 1.1 |
| (0, 2, 0) | 7835 | 11.7 | 3325 | 2.7 | 0.2 |
| (0, 2, 1) | 10000 | 14.9 | 7655 | 6.2 | 0.4 |
| (2, 0, −2) | 4131 | 6.2 | 16135 | 13.1 | 2.1 |
| (1, 2, 1) | 5251 | 7.8 | 20028 | 16.2 | 2.1 |
| (0, 1, 3) | 4806 | 7.2 | 5692 | 4.6 | 0.6 |
| (1, 1, −3) | 3305 | 4.9 | 10362 | 8.4 | 1.7 |
| (2, 2, 1) | 2560 | 3.8 | 715 | 0.6 | 0.2 |

*Calculated value of powder X-ray diffraction pattern calculated from single-crystal structure analysis data

### Example 5

Dissolution was performed with acetonitrile as a recrystallization solvent at 50° C. Then, when residual crystals were present, the crystals were removed. Cooling at room temperature was carried out to perform recrystallization, and filtration and air drying were performed to obtain crystals which were the anti-Acanthamoeba agent of the present invention. Powder X-ray analysis was performed in like manner with Example 2. The X-ray powder diffraction data of the resultant is as described below.

TABLE 5

| | Diffraction Peak | | | | |
|---|---|---|---|---|---|
| | Calculated Value* | | Crystal in Example 5 | | |
| | | Intensity | | Intensity | |
| | Intensity | Ratio | Intensity | Ratio | |
| Plane | (cps) | <A> (%) | (cps) | <B> (%) | B/A |
| (0, 0, 1) | 108 | 0.2 | −143 | −0.1 | −0.5 |
| (1, 0, 0) | 1741 | 2.6 | 705 | 0.4 | 0.2 |
| (1, 0, −1) | 1228 | 1.8 | 5505 | 3.3 | 1.8 |
| (0, 1, 1) | 2542 | 3.8 | 1283 | 0.8 | 0.2 |
| (1, 1, 0) | 765 | 1.1 | 237 | 0.1 | 0.1 |
| (1, 1, −1) | 9316 | 13.9 | 6498 | 3.8 | 0.3 |
| (1, 0, −2) | 5518 | 8.2 | 61310 | 36.3 | 4.4 |
| (1, 1, −2) | 7951 | 11.9 | 10385 | 6.1 | 0.5 |
| (0, 2, 0) | 7835 | 11.7 | 4942 | 2.9 | 0.2 |
| (0, 2, 1) | 10000 | 14.9 | 3280 | 1.9 | 0.1 |
| (2, 0, −2) | 4131 | 6.2 | 32428 | 19.2 | 3.1 |
| (1, 2, 1) | 5251 | 7.8 | 28663 | 17.0 | 2.2 |
| (0, 1, 3) | 4806 | 7.2 | 4432 | 2.6 | 0.4 |
| (1, 1, −3) | 3305 | 4.9 | 8698 | 5.1 | 1.0 |
| (2, 2, 1) | 2560 | 3.8 | 858 | 0.5 | 0.1 |

*Calculated value of powder X-ray diffraction pattern calculated from single-crystal structure analysis data

### Example 6

Dissolution was performed with methanol as a recrystallization solvent at 50° C., and cooling to 25° C. was carried out. Then, when residual crystals were present, the crystals were removed. Then, normal hexane was added as a poor solvent, recrystallization was performed at 25° C. under stirring, and filtration and air drying were performed to obtain crystals which were the anti-Acanthamoeba agent of the present invention. Powder X-ray analysis was performed in like manner with Example 2. The X-ray powder diffraction data of the resultant is as described below.

TABLE 6

| | Diffraction Peak | | | | |
|---|---|---|---|---|---|
| | Calculated Value* | | Crystal in Example 6 | | |
| | | Intensity | | Intensity | |
| | Intensity | Ratio | Intensity | Ratio | |
| Plane | (cps) | <A> (%) | (cps) | <B> (%) | B/A |
| (0, 0, 1) | 108 | 0.2 | 77 | 0.0 | 0.0 |
| (1, 0, 0) | 1741 | 2.6 | 905 | 0.4 | 0.2 |
| (1, 0, −1) | 1228 | 1.8 | 1302 | 0.6 | 0.3 |
| (0, 1, 1) | 2542 | 3.8 | 19055 | 9.2 | 2.4 |
| (1, 1, 0) | 765 | 1.1 | 527 | 0.3 | 0.3 |
| (1, 1, −1) | 0316 | 13.9 | 10525 | 5.1 | 0.4 |
| (1, 0, −2) | 5518 | 8.2 | 11075 | 5.3 | 0.6 |
| (1, 1, −2) | 7951 | 11.9 | 12243 | 5.9 | 0.5 |
| (0, 2, 0) | 7835 | 11.7 | 47283 | 22.8 | 1.9 |
| (0, 2, 1) | 10000 | 14.9 | 62990 | 30.4 | 2.0 |
| (2, 0, −2) | 4131 | 6.2 | 9785 | 4.7 | 0.8 |
| (1, 2, 1) | 5251 | 7.8 | 11717 | 5.7 | 0.7 |
| (0, 1, 3) | 4806 | 7.2 | 8530 | 4.1 | 0.6 |
| (1, 1, −3) | 3305 | 4.9 | 7523 | 3.6 | 0.7 |
| (2, 2, 1) | 2560 | 3.8 | 3637 | 1.8 | 0.5 |

*Calculated value of powder X-ray diffraction pattern calculated from single-crystal structure analysis data

### Example 7

The anti-Acanthamoeba activities of the crystals in Examples 2 to 6 were examined based on the method in Example 1. In other words, Acanthamoeba polyphaga (A. polyphaga) of which the number of the amoebae was 50,000 with respect to 4.5 mL of ATCC712 medium was dispersed and seeded in 500 μL of medium, and was preliminarily cultured at 25° C. for 48 hours. To the resultant, 1 mg of each of the crystals in Examples 2 to 6 was added to prepare a control and samples. The culture was further continued at 25° C. for 72 hours, and the number of the amoebae in the culture liquid was measured by a Neubauer counting chamber. The control, to which nothing was added, and in which the amoebae were cultured, was used.

Growth inhibition rates were calculated from the counted values according to an expression of [(the number of amoebae in control−the number of amoebae in each sample)/the number of amoebae in control]×100. This results are listed in Table 7.

Further, the average particle diameters of the crystals in Examples 2 to 6 were measured using an inverted microscope. In other words, three samples in each of the crystals of the examples were sampled. Using an inverted microscope Diaphot (40 times) manufactured by NIKON CORPORATION, the particle diameters of each sample were actually measured in 10 visual fields with a slide glass measure, and the average particle diameter thereof was measured. The particle diameters of the three samples were further averaged to obtain an average particle diameter. The results are listed in Table 7. The microphotographs of the crystals in Examples 2 to 6 are shown in FIG. 3.

TABLE 7

| | Average | | | | |
|---|---|---|---|---|---|
| | Particle | Growth | | | |
| | Diameter | Inhibition | | Peak Intensity Ratio (%) | |
| | (μm) | Rate (%) | SD | (10-2) Plane | (20-2) Plane |
| Crystal in | 232 | 66.7 | 2.8 | 27.4 | 15.5 |
| Example 2 | | | | | |
| Crystal in | 276 | 25.9 | 8.7 | 1.7 | 2.0 |
| Example 3 | | | | | |
| Crystal in | 213 | 69.6 | 1.5 | 24.3 | 13.1 |
| Example 4 | | | | | |
| Crystal in | 247 | 77.0 | 2.8 | 36.3 | 19.2 |
| Example 5 | | | | | |
| Crystal in | 183 | 54.8 | 11.4 | 5.3 | 4.7 |
| Example 6 | | | | | |

Examination of the relationship between the peak intensity ratio (%) of (10-2) plane and a growth inhibition rate (%), and the relationship between the peak intensity ratio (%) of (20-2) plane and a growth inhibition rate (%) based on the results described above revealed that the correlation coefficient of the former was 0.89 while the correlation coefficient of the latter was 0.90, exhibiting the favorable correlations. In other words, it is found that the peak intensity ratio of (20-2) plane is preferably 12% or more, more preferably 15% or more, and still more preferably 19% or more for crystals used in the anti-Acanthamoeba agent of the present invention. Further, it is found that the peak intensity ratio of (10-2) plane is preferably 20% or more, more preferably 25% or more, and still more preferably 30% or more. Further, it is found that it is preferable to satisfy both of such conditions.

### Example 8

Ophthalmic ointments were produced using the anti-Acanthamoeba agent of the present invention according to the following prescription. In other words, formulation ingredients were kneaded by a kneader under sterile conditions to obtain the ophthalmic ointments (unit: part(s) by mass).

TABLE 8

| | Ophthalmic | Ophthalmic | Ophthalmic | Ophthalmic | Ophthalmic |
|---|---|---|---|---|---|
| Ingredient | Ointment 1 | Ointment 2 | Ointment 3 | Ointment 4 | Ointment 5 |
| Vaseline | 99 | 99 | 99 | 99 | 99 |
| Crystal in | 1 | | | | |
| Example 2 | | | | | |
| Crystal in | | 1 | | | |
| Example 3 | | | | | |
| Crystal in | | | 1 | | |
| Example 4 | | | | | |
| Crystal in | | | | 1 | |
| Example 5 | | | | | |
| Crystal in | | | | | 1 |
| Example 6 | | | | | |

### Example 9

Ophthalmic powders were produced using the anti-Acanthamoeba agent of the present invention according to the following prescription. In other words, formulation ingredients were kneaded by a kneader under sterile conditions to obtain the ophthalmic powders (unit: part(s) by mass).

TABLE 9

| | Ophthalmic | Ophthalmic | Ophthalmic | Ophthalmic | Ophthalmic |
|---|---|---|---|---|---|
| Ingredient | Powder 1 | Powder 2 | Powder 3 | Powder 4 | Powder 5 |
| Maltitol | 99 | 99 | 99 | 99 | 99 |
| Crystal in | 1 | | | | |
| Example 2 | | | | | |
| Crystal in | | 1 | | | |
| Example 3 | | | | | |
| Crystal in | | | 1 | | |
| Example 4 | | | | | |
| Crystal in | | | | 1 | |
| Example 5 | | | | | |
| Crystal in | | | | | 1 |
| Example 6 | | | | | |

### Example 10

Pharmaceutical compositions in aqueous medium dispersant form were produced using the anti-Acanthamoeba agent of the present invention according to the following prescription. In other words, formulation ingredients were stirred to make homogeneous dispersion preparations, which were dispensed, sealed, and then sterilized in an autoclave (120° C., 15 minutes) to obtain the aqueous external preparations of the present invention (unit: percent(s) by mass).

TABLE 10

| | Aqueous | Aqueous | Aqueous | Aqueous | Aqueous |
|---|---|---|---|---|---|
| | Preparation | Preparation | Preparation | Preparation | Preparation |
| Ingredient | 1 | 2 | 3 | 4 | 5 |
| Water | 89 | 89 | 89 | 89 | 89 |
| 1,3-Butanediol | 9.7 | 9.7 | 9.7 | 9.7 | 9.7 |
| Methylparaben | 0.3 | 0.3 | 0.3 | 0.3 | 0.3 |
| Crystals in | 1 | | | | |
| Example 2 | | | | | |
| Crystals in | | 1 | | | |
| Example 3 | | | | | |
| Crystals in | | | 1 | | |
| Example 4 | | | | | |
| Crystals in | | | | 1 | |
| Example 5 | | | | | |
| Crystals in | | | | | 1 |
| Example 6 | | | | | |

### INDUSTRIAL APPLICABILITY

The present invention can be applied to a medicine.

1-4. (canceled)

5. A luliconazole crystal, having a crystal habit in which a ratio of I (20-2) to a total sum of I (001), I (100), I (10-1), I (011), I (110), I (11-1), I (10-2), I (11-2), I (020), I (021), I (20-2), I (121), I (013), I (11-3), and I (221) is 12% or more and/or a ratio of I (10-2) to a total sum of I (001), I (100), I (10-1), I (011), I (110), I (11-1), I (10-2), I (11-2), I (020), I (021), I (20-2), I (121), I (013), I (11-3), and I (221) is 20% or more;
provided that in diffraction peaks detected in a range of 2θ=5 to 35° in a powder X-ray diffraction pattern with CuKα as a radiation source, peak intensities at diffraction peaks corresponding to (001) plane, (100) plane, (10-1) plane, (011) plane, (110) plane, (11-1) plane, (10-2) plane, (11-2) plane, (020) plane, (021) plane, (20-2) plane, (121) plane, (013) plane, (11-3) plane, and (221) plane are I (001), I (100), I (10-1), I (011), I (110), I (11-1), I (10-2), I (11-2), I (020), I (021), I (20-2), I (121), I (013), I (11-3), and I (221), respectively.

6-7. (canceled)

8. A method for producing the luliconazole crystal as defined in claim 5, the method comprising recrystallizing luliconazole with acetonitrile that may contain a polar solvent; or recrystallizing luliconazole with a mixed solvent of an aprotic polar solvent and a protic polar solvent.

9. The production method according to claim 8, wherein the polar solvent is selected from water, methanol, ethanol, ethyl acetate, acetone, dimethylsulfoxide, and dimethylformamide.

10. (canceled)

11. The production method according to claim 8, wherein a solvent selected from normal hexane, cyclohexane, and petroleum ether is used as a poor solvent.

12. A method for treating an acanthamebiasis, comprising administering luliconazole to a subject in need thereof.

13. The method according to claim 12, wherein the luliconazole is present as a solid in an agent.

14. The method according to claim 12,
wherein the luliconazole has a crystal habit in which
a ratio of I (20-2) to a total sum of I (001), I (100), I (10-1), I (011), I (110), I (11-1), I (10-2), I (11-2), I (020), I (021), I (20-2), I (121), I (013), I (11-3), and I (221) is 12% or more; and/or a ratio of I (10-2) to a total sum of I (001), I (100), I (10-1), I (011), I (110), I (11-1), I (10-2), I (11-2), I (020), I (021), I (20-2), I (121), I (013), I (11-3), and I (221) is 20% or more;
provided that in diffraction peaks detected in a range of 2θ=5 to 35° in a powder X-ray diffraction pattern with CuKα as a radiation source, peak intensities at diffraction peaks corresponding to (001) plane, (100) plane, (10-1) plane, (011) plane, (110) plane, (11-1) plane, (10-2) plane, (11-2) plane, (020) plane, (021) plane, (20-2) plane, (121) plane, (013) plane, (11-3) plane, and (221) plane are I (001), I (100), I (10-1), I (011), I (110), I (11-1), I (10-2), I (11-2), I (020), I (021), I (20-2), I (121), I (013), I (11-3), and I (221), respectively.
